(19) United States
(12) Patent Application Publication
(10) Pub. No.: US 2019/0054215 A1
(43) Pub. Date: Feb. 21, 2019

(54) ARTIFICIAL CORNEAL STENT, AND ARTIFICIAL CORNEAL STROMA AND METHOD OF MAKING THE SAME

(71) Applicant: HON HAI PRECISION INDUSTRY CO., LTD., New Taipei (TW)

(72) Inventor: HSIU-WEN CHIEN, New Taipei (TW)

(21) Appl. No.: 15/692,328

(22) Filed: Aug. 31, 2017

(30) Foreign Application Priority Data
Aug. 15, 2017 (TW) 106127579

(51) Int. Cl. A61L 31/04; A61L 31/14; A61F 2/14; B29C 35/08; B29C 45/26; B29C 45/72; B29C 45/00

(52) U.S. Cl. CPC A61L 31/041; A61L 31/145; A61L 31/048; A61L 31/042; A61F 2/147; B29C 35/0805; B29L 2031/7546; B29C 45/72; B29C 45/0001; A61F 2240/001; A61F 2230/0004; A61F 2230/0069; B29C 45/263

(57) ABSTRACT

An artificial corneal stroma comprises a hydrogel substrate supported by artificial stents. At least one surface comprises a plurality of nanosize structures. The hydrogel substrate is formed from an artificial corneal stroma material. The artificial corneal stroma material comprises a methacrylate-bonded hyaluronic acid, a collagen, and a photoinitiator.

### FIELD

The subject matter relates to an artificial corneal stroma and a method of making the same, and an artificial corneal stent comprising the artificial corneal stroma.

### BACKGROUND

Cornea is a bloodless and optically transparent tissue. The cornea refracts and filters light entering the eye. The cornea includes five layers, which are epithelium, Bowman's membrane, stroma, Descemet's membrane, and endothelium, from the outside to the inside in that order. A corneal stent having the Bowman's membrane, the stroma, and the Descemet's membrane cannot be regenerated once damaged. There are a lot of cases of eye blindness caused by corneal diseases. Blindness caused by the corneal diseases can generally be cured by corneal transplantation surgeries, but human donors are few.

### BRIEF DESCRIPTION OF THE DRAWINGS

Implementations of the present technology will now be described, by way of example only, with reference to the attached figure, wherein:

FIG. 1 is a flowchart of a method for making an artificial corneal stent, in accordance with an exemplary embodiment of the present application.

FIG. 2 is a diagram of an artificial corneal stroma, in accordance with an exemplary embodiment of the present application.

FIG. 3A is a diagram of nanosize structures of surfaces of the artificial corneal stroma of FIG. 2, in accordance with an exemplary embodiment of the present application.

FIG. 3B is a diagram of nanosize structures of surfaces of the artificial corneal stroma of FIG. 2, in accordance with another exemplary embodiment of the present application.

FIG. 4 is a diagrammatic view of the nanosize structures of the artificial corneal stroma of FIG. 3, in accordance with an exemplary embodiment of the present application.

FIG. 5 is a diagram of an artificial corneal stent included in the artificial corneal stroma of FIG. 2, in accordance with an exemplary embodiment of the present application.

FIG. 6 is a flow diagram of method for manufacturing the artificial corneal stroma of FIG. 2, in accordance with an exemplary embodiment of the present application.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein can be practiced without these specific details.

In other instances, methods, procedures, and components have not been described in detail so as not to obscure the related relevant feature being described. Also, the description is not to be considered as limiting the scope of the exemplary embodiments described herein. The drawings are not necessarily to scale and the proportions of certain parts may be exaggerated to better illustrate details and features of the present disclosure.

One definition that applies throughout this disclosure will now be presented.

The term “substantially” is defined to be essentially conforming to the particular dimension, shape, or other feature that the term modifies, such that the component need not be exact. For example, “substantially rectangular” means that the object resembles a rectangle, but can have one or more deviations from a true rectangle.

The term “comprising,” when utilized, means “including, but not necessarily limited to”; it specifically indicates open-ended inclusion or membership in the so-described combination, assembly, series, and the like.

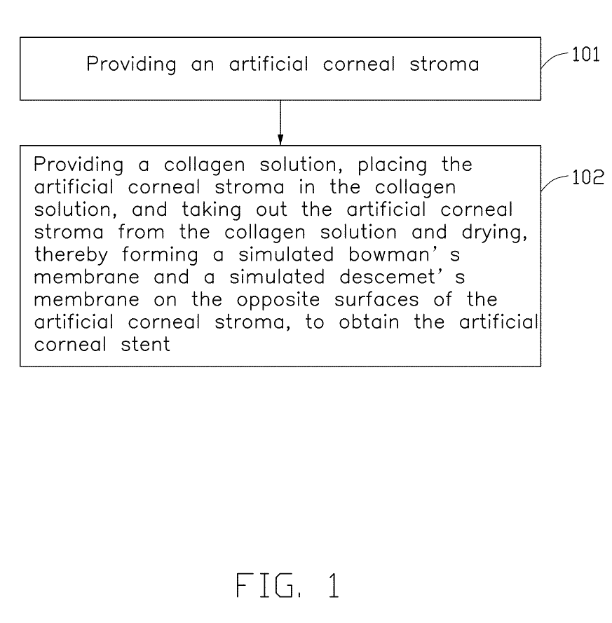

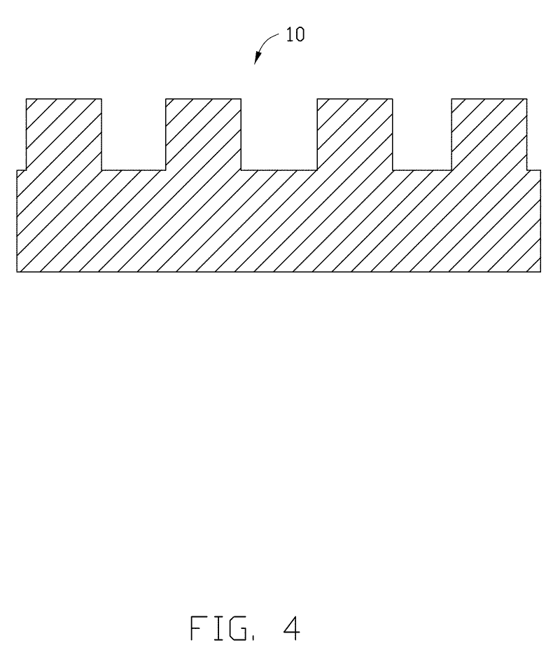

Referring to the FIG. 1, a method for making an artificial corneal stent 200 of FIG. 4 is illustrated. The exemplary method is provided by way of example, as there are a variety of ways to carry out the method. Each block shown in the figure represents one or more processes, methods, or subroutines, carried out in the exemplary method. Furthermore, the illustrated order of blocks is by example only, and the order of the blocks can change. Additional blocks may be added or fewer blocks may be utilized, without departing from this disclosure. The exemplary method can begin at block 101.

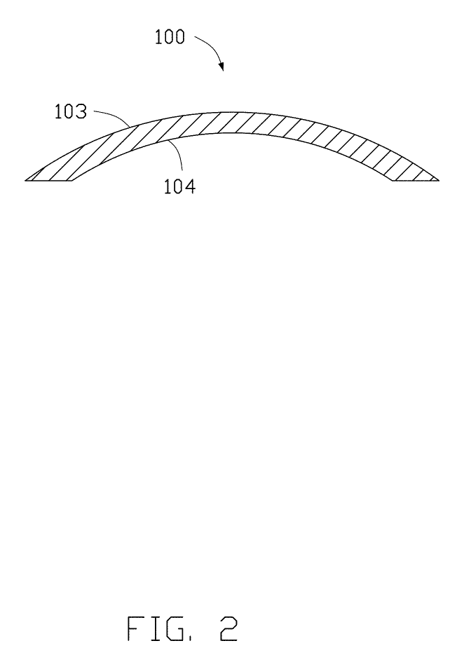

In block 101, referring to FIG. 2, an artificial corneal stroma 100 is provided.

The artificial corneal stroma 100 is made of an artificial corneal stroma material. The artificial corneal stroma 100 comprises an upper surface 103 and a bottom surface 104. The upper surface 103 and the bottom surface 104 are two opposite surfaces of the artificial corneal stroma 100.

In block 102, a collagen solution with a Bowman's membrane 201 is placed in a microfluid device, to simulate a Bowman's membrane 201 and a Descemet's membrane 202 with fiber orientations.

The artificial corneal stroma material comprises methacrylate-bonded hyaluronic acid, collagen, a photoinitiator, and a solvent. The methacrylate-bonded hyaluronic acid is formed by grafting methacrylate to the chain of the hyaluronic acid.

In the artificial corneal stroma material, a mass percentage of the methacrylate-bonded hyaluronic acid is in the range of 0.5% to 2.5%, a mass percentage of collagen is in the range of 0.1% to 1.5%, a mass percentage of the photoinitiator is in the range of 0.005% to 0.05%, and a mass percentage of the solvent is in the range of 96% to 98%.

A ratio of the molecular proportion of the methacrylate-bonded hyaluronic acid to the collagen is preferably 1:19 to 1:1.

The collagen comprises at least one of collagen type I, collagen type II, and collagen type III (trade names).

The photoinitiator comprises, but is not limited to, benzoin methyl ether, diethoxyacetophenone, benzoylphosphine oxide photoinitiator, 1-hydroxycyclohexyl phenyl ketone, Darocure type (chemical industry standard type) photoinitiator, and Irgacure type (chemical industry standard type) photoinitiator. The Darocure type photoinitiator can be Darocur-1173 or Darocur-2959. The Irgacure type photoinitiator can be Irgacure-1173 or Irgacure-2959. The benzoylphosphine oxide photoinitiator comprises 2,4,6-trimethylbenzoyldiphenylophosphine oxide, bis-(dichlorobenzoyl)-4-N-propylphenylphosphine oxide, bis-(2,6-dichlorobenzoyl)-4-N-butylphenylphosphine oxide. The photoinitiator and the methacrylate-bonded hyaluronic acid can polymerize methacrylate-bonded hyaluronic acid under ultraviolet irradiation, to form a cross-linking network structure.

The solvent may be acetic acid. The concentration of the acetic acid solution is about 0.05 mol/L.

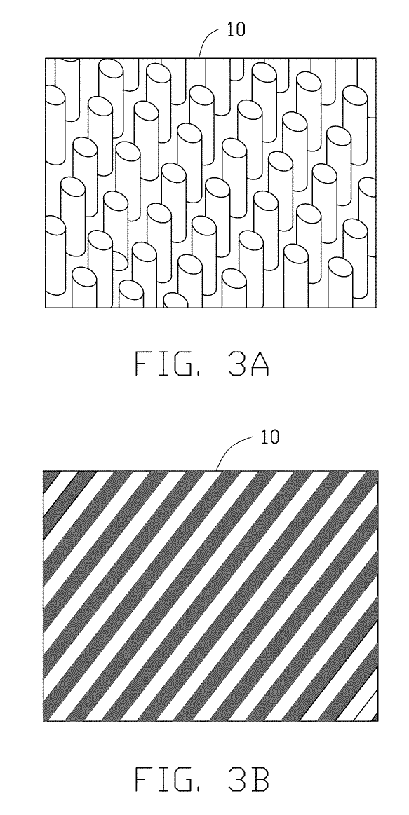

Referring to FIG. 3A, FIG. 3B, and FIG. 4, surfaces of the artificial corneal stroma 100 have a plurality of nanosize structures 10. The plurality of nanosize structures 10 may be columnar structures or stripe-shaped structures.

Referring to FIG. 3A and FIG. 4, when the nanosize structures 10 are columnar structures, the axis of each nanosize structure 10 is substantially perpendicular to the surfaces (e.g., the upper surface 103 and the bottom surface 104) of the artificial corneal stroma 100. Each nanosize structure 10 has a diameter in a range of 50 nanometers to 500 nanometers, and a height in a range of 5 nanometers to 500 nanometers. A distance between two adjacent nanosize structures 10 is in a range of 50 nanometers to 500 nanometers.

Referring to FIG. 3B and FIG. 4, when the nanosize structures are strip-shaped structures, each of two nanosize structures 10 are substantially parallel to each other. Each nanosize structure 10 has a width in a range of 50 nanometers to 500 nanometers, and a height in a range of 5 nanometers to 500 nanometers. A distance between two adjacent nanosize structures 10 is in a range of 50 nanometers to 500 nanometers.

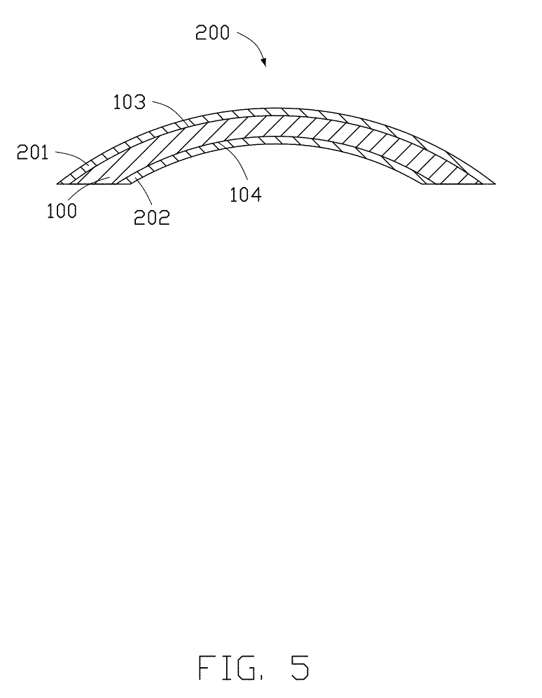

In block 102, referring to FIG. 1 and FIG. 5, a collagen solution is provided. The artificial corneal stroma 100 is placed in the collagen solution for about 5 minutes to about 60 minutes, and the artificial corneal stroma 100 is taken out from the collagen solution and dried, thereby forming a simulation of Bowman's membrane 201 on the upper surface 103 and a simulation Descemet's membrane 202 on the bottom surface 104, and the collagen of surfaces between the upper surface 103 and the bottom surface 104 is removed, to obtain the artificial corneal stent 200.

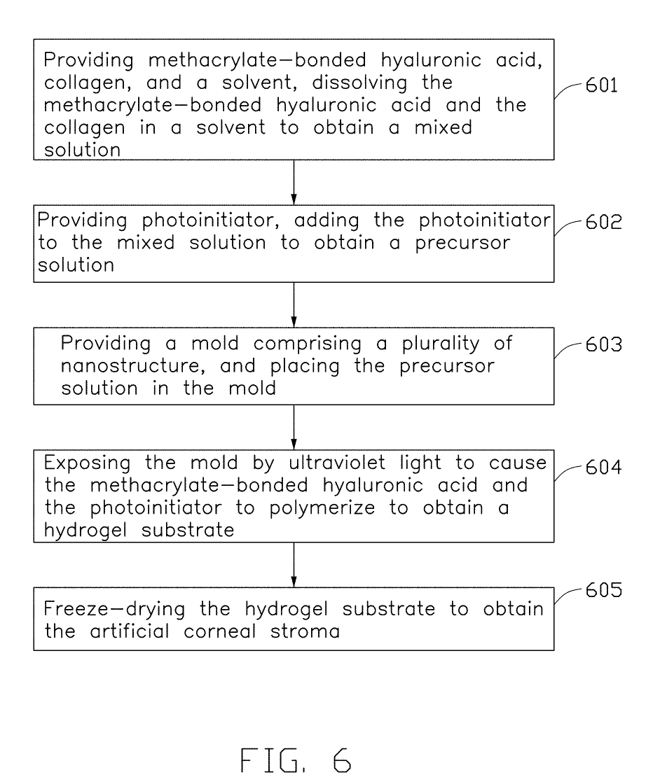

Referring to FIG. 6, a method for making the artificial corneal stroma 100 is illustrated. The exemplary method is provided by way of example, as there are a variety of ways to carry out the method. Each block shown in the figure represents one or more processes, methods, or subroutines, carried out in the exemplary method. Furthermore, the illustrated order of blocks is by example only, and the order of the blocks can change. Additional blocks may be added or fewer blocks may be utilized, without departing from this disclosure. The exemplary method can begin in block 601.

In block 601, the methacrylate-bonded hyaluronic acid and the collagenmethacrylate-bonded hyaluronic acid are dissolved in a solvent to obtain a mixed solution.

In block 602, the photoinitiator is provided, and the photoinitiator is added to the mixed solution to obtain a precursor solution.

In block 603, a mold is provided, and the precursor solution is placed in the mold.

The mold comprises a female die and a male die. In at least one embodiment, this step may be carried out by three stages: a mold opening stage, an injection stage, and a mold covering state.

The mold opening stage includes that the male die is separated from the female die.

The injection stage includes that the precursor solution is injected into the female die.

The mold covering stage includes that the female die is covered by the male die to define a space for accommodating the precursor solution.

In the exemplary embodiment, the space defined by the female die and the male die has a height of between 250 micrometers and 550 micrometers.

In the exemplary embodiment, a surface of the female die and a surface of the male die each has a number of nanostructures. The nanostructures control the arrangement of collagen cells in the artificial corneal stroma in cell culture stage. The nanostructures may comprise a plurality of cylindrical grooves. The shape of each cylindrical groove may be the same, and a distance between adjacent two cylindrical grooves may be the same. Each nanosize structure may also be stripe-shaped.

In block 604, the mold is exposed to ultraviolet irradiation to cause the methacrylate-bonded hyaluronic acid and the photoinitiator to polymerize to form a cross-linking network structure, thereby obtaining a hydrogel substrate.

In block 605, the hydrogel substrate is demolded and washed.

In block 606, the demolded and washed hydrogel is freeze-dried to obtain the artificial corneal stroma 100.

The shape of the artificial corneal stroma 100 can be adjusted as needed. In the exemplary embodiment, the artificial corneal stroma 100 has a shape of a contact lens.

Referring to FIG. 5, the artificial corneal stent 200 comprises the artificial corneal stroma 100, the simulated Bowman's membrane 201, and the simulated Descemet's membrane 202. The membrane 201 and the membrane 202 are formed on the opposite surfaces of the artificial corneal stroma 100. Membranes 201 and 202 are made of collagen.

Since the artificial corneal stroma material comprises methacrylate-bonded hyaluronic acid and the photoinitiator, the methacrylate-bonded hyaluronic acid and the photoinitiator can be polymerized to form a cross-linking network structure under ultraviolet radiation, thus forming the artificial corneal stroma. The surfaces of the female die and the male die have nanostructures, which control the arrangement of cells in the artificial corneal stroma in the cell culture stage.

The embodiments shown and described above are only examples. Many details are often found in the art such as the other features. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. An artificial corneal stroma comprising:
a hydrogel substrate, the hydrogel substrate having at least one surface comprising a plurality of nanosize structures;
wherein the hydrogel substrate is formed by an artificial corneal stroma material, the artificial corneal stroma material comprises a methacrylate-bonded hyaluronic acid, a collagen, and a photoinitiator.

2. The artificial corneal stroma of claim 1, wherein the plurality of nanosize structures comprises columnar structures, the axis of at least one of the plurality of nanosize structures is perpendicular to the at least one surface of the artificial corneal stroma.

3. The artificial corneal stroma of claim 2, wherein at least one of the plurality of nanosize structures has a diameter in a range of 50 nanometers to 500 nanometers, and a height in a range of 5 nanometers to 500 nanometers, a distance between two adjacent ones of the plurality of nanosize structures is 50 nanometers to 500 nanometers.

4. The artificial corneal stroma of claim 1, wherein the plurality of nanosize structures are strip-shaped, at least two of the plurality of nanosize structures are parallel.

5. The artificial corneal stroma of claim 4, wherein at least one of the plurality of the nanosize structures has a width in a range of 50 nanometers to 500 nanometers, and a height in a range of 5 nanometers to 500 nanometers, and a distance between two adjacent ones of the plurality of nanosize structures is in a range of 50 nanometers to 500 nanometers.

6. The artificial corneal stroma of claim 1, wherein a ratio of molecular proportion of the methacrylate-bonded hyaluronic acid to the collagen is 1:19 to 1:1.

7. The artificial corneal stroma of claim 6, wherein a mass percentage of the methacrylate-bonded hyaluronic acid is in a range of 0.5% to 2.5%, a mass percentage of collagen is in a range of 0.1% to 1.5%, a mass percentage of the photoinitiator is in a range of 0.005% to 0.05%, and a mass percentage of the solvent is in a range of 96% to 98%.

8. A method of making the artificial corneal stroma comprising:
providing methacrylate-bonded hyaluronic acid, collagen, and a solvent, dissolving the methacrylate-bonded hyaluronic acid and the collagen in a solvent to obtain a mixed solution;
providing photoinitiator, adding the photoinitiator to the mixed solution to obtain a precursor solution;
providing a mold comprising a plurality of nanosize structures, and placing the precursor solution in the mold;
exposing the mold by ultraviolet light to cause the methacrylate-bonded hyaluronic acid and the photoinitiator to polymerize to obtain a hydrogel substrate; and
freeze-drying the hydrogel substrate to obtain the artificial corneal stroma.

9. The method of claim 8, wherein the plurality of nanosize structures comprise a plurality of cylindrical grooves.

10. The method of claim 8, wherein at least one of the a plurality of nanosize structures is stripe-shaped.

11. An artificial corneal stent, the artificial corneal stent comprising:
an artificial corneal stroma, the artificial corneal stroma comprising a hydrogel substrate, at least one surface of the hydrogel substrate comprising a plurality of nanosize structures;
a simulated bowman's membrane; and
a simulated descemet's membrane;
wherein the simulated bowman's membrane and the simulated descemet's membrane are formed on opposite surfaces of the artificial corneal stroma, the simulated bowman's membrane and the simulated descemet's membrane are made of collagen.

12. The artificial corneal stent of claim 11, wherein the plurality of nanosize structures comprises columnar structures, the axis of at least one of the plurality of nanosize structures is perpendicular to at least one of the opposite surfaces of the artificial corneal stroma.

13. The artificial corneal stent of claim 12, wherein at least one of the plurality of nanosize structures has a diameter in a range of 50 nanometers to 500 nanometers, and a height in a range of 5 nanometers to 500 nanometers, a distance between two adjacent ones of the plurality of nanosize structures is in a range of 50 nanometers to 500 nanometers.

14. The artificial corneal stent of claim 11, wherein the plurality of nanosize structures are strip-shaped, at least one of the plurality of nanosize structures extends in a direction parallel to at least one of the opposite surfaces of the artificial corneal stroma.

15. The artificial corneal stent of claim 14, wherein at least one of the plurality of the nanosize structures has a width in a range of 50 nanometers to 500 nanometers, and a height in a range of 5 nanometers to 500 nanometers, a distance between two adjacent ones of the plurality of nanosize structures is in a range of 50 nanometers to 500 nanometers.

16. The artificial corneal stent of claim 11, wherein a ratio of molecular proportion of the methacrylate-bonded hyaluronic acid to the collagen is 1:19 to 1:1.

17. The artificial corneal stent of claim 16, wherein a mass percentage of the methacrylate-bonded hyaluronic acid is in a range of 0.5% to 2.5%, a mass percentage of collagen is in a range of 0.1% to 1.5%, a mass percentage of the photoinitiator is in a range of 0.005% to 0.05%, and a mass percentage of the solvent is in a range of 96% to 98%.
